(19) United States
(12) Patent Application Publication
(10) Pub. No.: US 2003/0096377 A1
(43) Pub. Date: May 22, 2003

(54) Differential PCR-RFLP assay for detecting and distinguishing between nonpathogenic PCV-1 and pathogenic PCV-2

(75) Inventors: Xiang-Jin Meng, Blacksburg, VA (US); Martijn Fenaux, Blacksburg, VA (US)

Correspondence Address: ANNE M. ROSENBLUM, ESQ., Suite 212, 163 Delaware Avenue, Delmar, NY, 12054, US

(73) Assignee: Virginia Tech Intellectual Properties, Inc., Blacksburg, VA (US)

(21) Appl. No.: 10/184,191

(22) Filed: Jun. 27, 2002

Related U.S. Application Data
(60) Provisional application No. 60/301,707, filed on Jun. 28, 2001.

(51) Int. Cl. C12Q 1/70; C12Q 1/68; C12P 19/34

(52) U.S. Cl. 435/91.2; 435/5; 435/6; 435/91.1

(57) ABSTRACT

The present invention relates to a method for detecting and differentiating PCV infections in a biological sample taken from a pig which involves amplifying a fragment from an extracted nucleic acid; digesting the fragment with a suitable restriction enzyme such as the unique NcoI restriction enzyme; forming a restriction fragment length polymorphism pattern; and then detecting the presence or absence of a PCV isolate. The invention further concerns the new oligonucleotide primers for differentiating PCV infections comprising a nucleotide sequence selected from the group consisting of MCV1 having a nucleotide sequence set forth in SEQ ID NO: 1 and MCV2 having a nucleotide sequence set forth in SEQ ID NO: 2. Moreover, this invention provides a novel kit for detecting and distinguishing PCV infections that includes the new oligonucleotide primers and the suitable restriction enzyme.

### CROSS-REFERENCE TO RELATED U.S. APPLICATIONS

This application claims the benefit under 35 U.S.C. § 119 (e) of U.S. Provisional Application No. 60/301,707, filed Jun. 28, 2001. The prior application is incorporated herein by reference in its entirety.

### STATEMENT REGARDING FEDERALLY SPONSORED RESEARCH OR DEVELOPMENT

Not Applicable

### REFERENCE TO A “Sequence Listing”

The material on a single compact disc containing a Sequence Listing file provided in this application is incorporated by reference. The date of creation is ______, 2002 and the size is ______.

### BACKGROUND OF THE INVENTION

1. Field of the Invention

The present invention concerns a method for detecting and distinguishing porcine circovirus (PCV) infections by a novel differential polymerase chain reaction-restriction fragment length polymorphism (PCR-RFLP) assay using a restriction enzyme and new primers. The invention further relates to a kit for detecting and distinguishing PCV infections that includes the restriction enzyme and the primers.

2. Description of the Related Art

All patents and publications cited in this specification are incorporated herein by reference in their entirety.

Porcine circovirus (PCV) was originally isolated as a noncytopathic contaminant of the porcine kidney cell line, PK-15 (I. Tischer et al., “A very small porcine virus with circular single-stranded DNA,” Nature 295:64-66 (1982)). PCV is a small nonenveloped virus that contains a single-stranded circular DNA genome of about 1.76 kb (id.). Based on the morphology and genomic organization, PCV was classified as a member of Circoviridae family (P. D. Lukert et al., “The Circoviridae,” pp. 166-168, In: Virus Taxonomy: sixth report of the International Committee on Taxonomy of Viruses, F. A. Murphy et al., eds, Springer-Verlag, Vienna, Austria (1995); B. M. Meehan et al., “Sequence of porcine circovirus DNA: affinities with plant circoviruses,” J. Gen. Virol. 78:221-227 (1997)), which consists of two other animal circoviruses: chicken anemia virus (CAV) and psittacine beak-and-feather disease virus (PBFDV) and three plant circoviruses: banana bunchy top virus (BBTV), coconut foliar decay virus (CFDV) and subterranean clover stunt virus (SCSV). Members of the three recognized animal circoviruses, PCV, CAV and PBFDV, do not share nucleotide sequence homology or antigenic determinants with each other (M. R. Bassami et al., “Psittacine beak and feather disease virus nucleotide sequence analysis and its relationship to porcine circovirus, plant circoviruses, and chicken anaemia virus,” Virology 249:453-459 (1998); D. Todd et al., “Comparison of three animal viruses with circular single-stranded DNA genomes,” Arch. Virol. 117:129-135 (1991)). More recently, a human circovirus, designated as TT virus (TTV), was identified from individuals with posttransfusion hepatitis (H. Miyata et al., “Identification of a novel GC-rich 113-nucleotide region to complete the circular, single-stranded DNA genome of TT virus, the first human circovirus,” J. Virol. 73:3582-3586 (1999); T. Nishizawa et al., “A novel DNA virus (TTV) associated with elevated transaminase levels in posttransfusion hepatitis of unknown etiology,” Biochem. Biophys. Res. Commun. 241:92-97 (1997)). The human TTV is similar to the circovirus CAV in its genomic organization (H. Miyata et al., 1999, supra). Although antibodies to PCV were found in various animal species including humans, mice, cattle and pigs (G. C. Dulac et al., “Porcine circovirus antigens in PK-15 cell line (ATCC CCL-33) and evidence of antibodies to circovirus in Canadian pigs,” Can. J. Vet. Res. 53:431-433 (1989); S. Edwards et al., “Evidence of circovirus infection in British pigs,” Vet. Rec. 134:680-1 (1994); R. K. Hines et al., “Porcine circovirus: a serological survey of swine in the United States,” Journal of Swine Health and Production 3:71-73 (1995); R. K. Hines et al., “Some effects of porcine circovirus on performance,” Journal of Swine Health and Production 3:251-255 (1995); G. P. Nayar et al., “Evidence for circovirus in cattle with respiratory disease and from aborted bovine fetuses,” Can. Vet. J. 40:277-278 (1999); I. Tischer et al., “Distribution of antibodies to porcine circovirus in swine populations of different breeding farms,” Arch. Virol. 140:737-743 (1995); I. Tischer et al., “Presence of antibodies reacting with porcine circovirus in sera of humans, mice, and cattle,” Arch. Virol. 140:1427-1439 (1995)), little is known regarding the pathogenesis of PCV in these animal species. Experimental infection of pigs with the PK-15-derived PCV did not produce clinical disease and thus, this virus is not considered to be pathogenic to pigs (G. M. Allan et al., “Pathogenesis of porcine circovirus; experimental infections of colostrum deprived piglets and examination of pig foetal material,” Vet. Microbiol. 44:49-64 (1995); I. Tischer et al., “Studies on epidemiology and pathogenicity of porcine circovirus,” Arch. Virol. 91:271-276 (1986)).

Postweaning multisystemic wasting syndrome (PMWS) is a new and unique disease in pigs, which was first described in 1991 (E. G. Clark, “Postweaning multisystemic wasting syndrome,” Proceedings of American Association of Swine Practitioners, pp. 499-501 (1997); J. C. Harding, “Postweaning multisystemic wasting syndrome (PMWS): preliminary epidemiology and clinical presentation,” p. 503, In Proceedings of American Association of Swine Practitioners, American Association of Swine Practitioners, Quebec City, Canada (1997)). Since the initial recognition of the disease, PMWS has emerged to be an economically important global disease of swine (G. M. Allan et al., “Novel porcine circoviruses from pigs with wasting disease syndromes,” Vet. Rec. 142:467-468 (1998); G. M. Allan et al., “Isolation of porcine circovirus-like viruses from pigs with a wasting disease in the USA and Europe,” J. Vet. Diagn. Invest. 10:3-10 (1998); G. M. Allan et al., “Isolation and characterisation of circoviruses from pigs with wasting syndromes in Spain, Denmark and Northern Ireland,” Vet. Microbiol. 66:115-23 (1999); G. M. Allan et al., “Porcine circoviruses: a review,” J. Vet. Diagn. Invest. 12:3-14 (2000); J. A. Ellis et al., “Isolation of circovirus from lesions of pigs with postweaning multisystemic wasting syndrome,” Can. Vet. J. 39:44-51 (1998); A. L. Hamel et al., “Nucleotide sequence of porcine circovirus associated with postweaning multisystemic wasting syndrome in pigs,” J. Virol. 72:5262-5267 (1998); S. Kennedy et al., “Porcine circovirus infection in Northern Ireland,” Vet. Rec. 142:495-6 (1998); M. Kiupel et al., “Circovirus-like viral associated disease in weaned pigs in Indiana,” Vet. Pathol. 35:303-307 (1998); R. Larochelle et al., “Identification and incidence of porcine circovirus in routine field cases in Quebec as determined by PCR,” Vet. Rec. 145:140-142 (1999); A. Mankertz el al., “Characterisation of PCV-2 isolates from Spain, Germany and France,” Virus Res. 66:65-77 (2000); B. M. Meehan et al., “Characterization of novel circovirus DNAs associated with wasting syndromes in pigs,” J. Gen. Virol. 79:2171-2179 (1998); I. Morozov et al., “Detection of a novel strain of porcine circovirus in pigs with postweaning multisystemic wasting syndrome,” J. Clin. Microbiol. 36:2535-2541 (1998); G. P. Nayar et al., “Detection and characterization of porcine circovirus associated with postweaning multisystemic wasting syndrome in pigs,” Can. Vet. J. 38:385-386 (1997); A. Onuki et al., “Detection of porcine circovirus from lesions of a pig with wasting disease in Japan,” J. Vet. Med. Sci. 61:1119-1123 (1999); J. Segales et al., “First report of postweaning multisystemic wasting syndrome in Spain,” Vet. Rec. 141:600-601 (1997); P. Spillane et al., “Porcine circovirus infection in the Republic of Ireland,” Vet. Rec. 143:511-512 (1998)). The disease occurs in high-health swine herds as a low morbidity but high case fatality disease of 5- to 12-week-old pigs (G. M. Allan et al., 2000, supra; E. G. Clark,, 1997, supra). Clinically, PMWS is characterized by progressive weight loss, dyspnea, tachypnea, anemia, diarrhea and jaundice. In an acute outbreak, the mortality rate associated with PMWS may peak at about 10% and can reach up to 50% in some cases (G. M. Allan et al., 2000, supra; J. C. Harding, “Postweaning multisystemic wasting syndrome (PMWS): preliminary epidemiology and clinical presentation,” p. 503, In Proceedings of American Association of Swine Practitioners, American Association of Swine Practitioners, Quebec City, Canada (1997); J. C. Harding et al., “Recognizing and diagnosing postweaning multisystemic wasting syndrome (PMWS),” Journal of Swine Health and Production 5:201-203 (1997)). Pathologically, microscopic lesions of PMWS include granulomatous interstitial pneumonia, lymphadenopathy, hepatitis, nephritis and pancreatitis (J. C. Harding, “Postweaning multisystemic wasting syndrome (PMWS): preliminary epidemiology and clinical presentation,” p. 503, In Proceedings of American Association of Swine Practitioners, American Association of Swine Practitioners, Quebec City, Canada (1997); J. C. Harding et al., “Recognizing and diagnosing postweaning multisystemic wasting syndrome (PMWS),” Journal of Swine Health and Production 5:201-203 (1997)). PMWS has now been recognized in pigs in Canada and most of the United States (G. M. Allan et al., “Novel porcine circoviruses from pigs with wasting disease syndromes,” Vet. Rec. 142:467-468 (1998); G. M. Allan et al., “Isolation of porcine circovirus-like viruses from pigs with a wasting disease in the USA and Europe,” J. Vet. Diagn. Invest. 10:3-10 (1998); G. M. Allan et al., 2000, supra; J. A. Ellis et al., 1998, supra; A. L. Hamel et al., 1998, supra; M. Kiupel et al., 1998, supra; R. Larochelle et al., “Identification and incidence of porcine circovirus in routine field cases in Quebec as determined by PCR,” Vet. Rec. 145:140-142 (1999); B. M. Meehan et al., 1998, supra; I. Morozov et al., 1998, supra; G. P. Nayar et al., 1997, supra), many European countries (G. M. Allan et al., “Isolation and characterisation of circoviruses from pigs with wasting syndromes in Spain, Denmark and Northern Ireland,” Vet. Microbiol. 66:115-23 (1999); G. M. Allan et al., 2000, supra; S. Kennedy et al., 1998, supra; A. Mankertz et al., 2000, supra; J. Segales et al., 1997, supra; P. Spillane et al., 1998, supra) and some countries in Asia (G. M. Allan et al., 2000, supra; A. Onuki et al., 1999, supra), and potentially has serious economic impact on swine industry worldwide.

The etiology of PMWS is very complicated (C. Rosell et al., “Identification of porcine circovirus in tissues of pigs with porcine dermatitis and nephropathy syndrome,” Vet. Rec. 146:40-43 (2000)), but it is believed that a variant strain of PCV, designated as type-2 PCV (PCV-2), is responsible for PMWS in pigs (G. M. Allan et al., “Novel porcine circoviruses from pigs with wasting disease syndromes,” Vet. Rec. 142:467-468 (1998); G. M. Allan et al., “Isolation of porcine circovirus-like viruses from pigs with a wasting disease in the USA and Europe,” J. Vet. Diagn. Invest. 10:3-10 (1998); G. M. Allan et al., “Isolation and characterisation of circoviruses from pigs with wasting syndromes in Spain, Denmark and Northern Ireland,” Vet. Microbiol. 66:115-23 (1999); G. M. Allan et al., 2000, supra; J. A. Ellis et al., 1998, supra; A. L. Hamel et al., 1998, supra; B. M. Meehan et al., 1998, supra; I. Morozov et al., 1998, supra). The nonpathogenic PK-15-derived PCV has been designated as PCV-1 to distinguish it from the PMWS-associated PCV-2 (G. M. Allan et al., “Novel porcine circoviruses from pigs with wasting disease syndromes,” Vet. Rec. 142:467-468 (1998); G. M. Allan et al., “Isolation of porcine circovirus-like viruses from pigs with a wasting disease in the USA and Europe,” J. Vet. Diagn. Invest. 10:3-10 (1998)). PCV-2 was isolated from pigs with clinical and pathological findings consistent with PMWS (G. M. Allan el al., “Novel porcine circoviruses from pigs with wasting disease syndromes,” Vet. Rec. 142:467-468 (1998); G. M. Allan et al., “Isolation of porcine circovirus-like viruses from pigs with a wasting disease in the USA and Europe,” J. Vet. Diagn. Invest. 10:3-10 (1998); G. M. Allan et al., “Isolation and characterisation of circoviruses from pigs with wasting syndromes in Spain, Denmark and Northern Ireland,” Vet. Microbiol. 66:115-23 (1999); G. M. Allan et al., 2000, supra; J. A. Ellis et al., 1998, supra; A. L. Hamel et al., “PCR detection and characterization of type-2 porcine circovirus,” Can. J. Vet. Res. 64:44-52 (2000); R. Larochelle et al., “Identification and incidence of porcine circovirus in routine field cases in Quebec as determined by PCR,” Vet. Rec. 145:140-142 (1999); 39; G. P. Nayar et al., 1997, supra). PCV-2 DNA and antigen have been detected in various tissues and organs from natural cases of PMWS in pigs (A. L. Hamel et al., 2000, supra; R. Larochelle et al., “Identification and incidence of porcine circovirus in routine field cases in Quebec as determined by PCR,” Vet. Rec. 145:140-142 (1999); A. Mankertz et al., 2000, supra; B. M. Meehan et al., 1998, supra; I. Morozov el al., 1998, supra; G. P. Nayar et al., 1997, supra; C. Rosell et al., “Pathological, immunohistochemical, and in-situ hybridization studies of natural cases of postweaning multisystemic wasting syndrome (PMWS) in pigs,” J. Comp. Pathol. 120:59-78 (1999)).

Many known swine pathogens such as porcine reproductive and respiratory syndrome virus (PRRSV), swine influenza virus, hemagglutinating encephalomyocarditis virus, porcine proliferative enteropathy, M. hypopneumoniae, H. parasuis, postweaning colibacillosis, etc. could cause postweaning wasting of pigs (J. C. Harding et al., “Recognizing and diagnosing postweaning multisystemic wasting syndrome (PMWS),” Journal of Swine Health and Production 5:201-203 (1997)). However, increasing data indicate that PCV-2 is the causative agent of PMWS (G. M. Allan et al., “Novel porcine circoviruses from pigs with wasting disease syndromes,” Vet. Rec. 142:467-468 (1998); G. M. Allan et al., “Isolation of porcine circovirus-like viruses from pigs with a wasting disease in the USA and Europe,” J. Vet. Diagn. Invest. 10:3-10 (1998); G. M. Allan et al., “Isolation and characterisation of circoviruses from pigs with wasting syndromes in Spain, Denmark and Northern Ireland,” Vet. Microbiol. 66:115-23 (1999); G. M. Allan et al., 2000, supra; J. A. Ellis et al., 1998, supra; A. L. Hamel et al., 1998, supra; B. M. Meehan et al., 1998, supra; I. Morozov et al., 1998, supra). Experimental inoculation of conventional pigs with tissue homogenates from pigs exhibiting clinical signs of PMWS produced PMWS-like lesions, and PCV-2 DNA and antibody to PCV-2 were detected in the inoculated pigs (M. Balasch et al., “Experimental inoculation of conventional pigs with tissue homogenates from pigs with post-weaning multisystemic wasting syndrome,” J. Comp. Pathol. 121:139-148 (1999)). Ellis et al. experimentally inoculated neonatal gnotobiotic piglets with filtered tissue culture materials and tissue homogenates from PMWS-affected pigs (J. A. Ellis et al., “Reproduction of lesions of postweaning multisystemic wasting syndrome in gnotobiotic piglets,” J. Vet. Diagn. Invest. 11:3-14 (1999)). The inoculated gnotobiotic piglets developed lesions typical of PMWS, but the study was complicated by the detection of porcine parvovirus (PPV) in inoculated piglets. In fact, coinfection by PPV and PCV in pigs with naturally acquired PMWS has been reported (J. A. Ellis et al., “Coinfection by porcine circoviruses and porcine parvovirus in pigs with naturally acquired postweaning multisystemic wasting syndrome,” J. Vet. Diagn. Invest. 12:21-27 (2000)). It has also been shown that PCV-2 alone induced PMWS lesions in colostrum-deprived conventional pigs but concurrent infection with PPV increased the severity of the lesions (G. M. Allan et al., “Experimental reproduction of severe wasting disease by co-infection of pigs with porcine circovirus and porcine parvovirus,” J. Comp. Pathol. 121:1-11 (1999); S. Kennedy et al., “Reproduction of lesions of postweaning multisystemic wasting syndrome by infection of conventional pigs with porcine circovirus type 2 alone or in combination with porcine parvovirus,” J. Comp. Pathol. 122:9-24 (2000)), suggesting that PMWS is a complex disease syndrome and multi-factors may be involved in the pathogenesis of PMWS. It has been suspected that some of the clinical signs and pathological lesions attributable to PRRSV may actually be induced by PCV-2 as a result of PCV-2 infection or coinfection (J. A. Ellis, “‘The clinical scope of porcine reproductive and respiratory syndrome virus infection has expanded since 1987’: an alternative perspective,” Vet. Pathol. 36:262-264 (1999); R. Larochelle et al., “Identification and incidence of porcine circovirus in routine field cases in Quebec as determined by PCR,” Vet. Rec. 145:140-142 (1999)). Synergism between a circovirus (CAV) and a reovirus was observed following dual infection of chickens by a natural route (F. McNeilly et al., “Synergism between chicken anemia virus (CAV) and avian reovirus following dual infection of 1-day-old chicks by a natural route,” Avian Dis. 39:532-537 (1995)).

The complete genome of PCV-2 has been determined and, interestingly, the nonpathogenic PCV-1 and the PMWS-associated PCV-2 are found to share only about 75% nucleotide sequence identity (A. L. Hamel et al., 1998, supra; B. M. Meehan et al., 1998, supra; I. Morozov et al., 1998, supra). Seven open reading frames (ORFs) have been identified for PCV-1 (A. Mankertz et al., “Mapping and characterization of the origin of DNA replication of porcine circovirus,” J. Virol. 71:2562-2566 (1997); J. Mankertz et al., “Transcription analysis of porcine circovirus (PCV),” Virus Genes 16:267-276 (1998); B. M. Meehan et al., 1997, supra) whereas thirteen ORFs have now been identified for PCV-2 (Bublot et al., WO 00/77216 A2). Previously, six to eleven ORFs had been identified for PCV-2 (A. L. Hamel et al., 1998, supra; B. M. Meehan et al., 1998, supra; I. Morozov et al., 1998, supra). Although PMWS has been reported in most of the United States, only a few PCV-2 isolates from the U.S. have been genetically characterized (B. M. Meehan et al., 1998, supra; I. Morozov et al., 1998, supra). Based on the nucleotide sequence of the U.S. and other PCV-2 isolates sequenced thus far, it appears that there exists only one genotype of PCV-2 worldwide (A. L. Hamel et al., 2000, supra; B. M. Meehan et al., 1998, supra; I. Morozov et al., 1998, supra).

Since PCV-1 is nonpathogenic and widespread in pig population, a test is needed to differentiate between infections with PCV-1 and PCV-2. In addition, since antibody to PCV has been detected in humans (I. Tischer et al., “Presence of antibodies reacting with porcine circovirus in sera of humans, mice, and cattle,” Arch. Virol. 140:1427-1439 (1995)), a major and growing concern is the inadvertent transmission of PCV from pig organs to human recipients during xenotransplantation. In xenotransplantation, there is zero tolerance for circovirus infection regardless of its pathogenic potential, since nonpathogenic PCV-1 may become pathogenic in immunocompromised xenograft recipients. Among other numerous present and potential uses, fetal pig brain cells are being injected, for example, into a human's brain to reverse certain forms of paralysis caused by stroke. Not only does the patient risk developing a potentially fatal pig viral disease but the patient also must be forever careful not to transmit contagious pig viral infections to others. In addition to a clear benefit to stroke victims, a wider range of potential medical and veterinary treatments may become more practical if the threat of pig infection was significantly reduced. Therefore, rapid, sensitive and easy-to-perform assays are needed to screen for both PCV-1 and PCV-2 infection in xenograft donor pigs.

Several techniques such as polymerase chain reaction (PCR) (A. L. Hamel et al., 2000, supra; R. Larochelle et al., “Identification and incidence of porcine circovirus in routine field cases in Quebec as determined by PCR,” Vet. Rec. 145:140-142 (1999); R. Larochelle et al., “Typing of porcine circovirus in clinical specimens by multiplex PCR,” J. Virol. Methods 80:69-75 (1999); I. Morozov et al., 1998, supra; G. P. Nayar et al., 1997, supra; M. Ouardani et al., “Multiplex PCR for detection and typing of porcine circoviruses,” J. Clin. Microbiol. 37(12):3917-24 (1999)), immunohistochemistry (IHC) (F. McNeilly et al., “A comparison of in situ hybridization and immunohistochemistry for the detection of a new porcine circovirus in formalin-fixed tissues from pigs with post-weaning multisystemic wasting syndrome (PMWS),” J. Virol. Methods 80:123-128 (1999); I. Morozov et al., 1998, supra; C. Rosell et al., 1999, supra), in situ hybridization (C. Choi et al., “In-situ hybridization for the detection of porcine circovirus in pigs with postweaning multisystemic wasting syndrome,” J. Comp. Pathol. 121:265-270 (1999); F. McNeilly et al., 1999, supra; I. Morozov et al., 1998, supra; C. Rosell et al., 1999, supra) are available for detecting PCV-2 infection. Nayar et al., without much description, report employing a PCR test, which was modified for the detection of PCV DNA, on tissue samples of sick pigs showing clinical signs and pathology consistent with PMWS but out of 100 pigs tested, they only found 15 cases positive for PCV DNA (G. P. Nayar et al., 1997, supra). Using a restriction enzyme (RE) cleavage map analysis, Nayar et al. conclude that the PCV from pigs with PMWS will possess different RE types than PCV isolated from other samples and which were nonpathogenic like that of PCV from PK-15 cell lines. The PCV from the PK-15 isolate and the PCV from pigs suffering from PMWS were thereafter characterized in more detail and named PCV-1 and PCV-2, respectively (G. M. Allan et al., “Novel porcine circoviruses from pigs with wasting disease syndromes,” Vet. Rec. 142:467-468 (1998); 3). Allan et al. (WO 99/18214) describe the detection of cell cultures containing PCV-2 by immunofluorescence or in situ hydridization. Allan et al. (U.S. Pat. No. 6,217,883) further disclose the detection of PCV or the genes thereof by standard methods known in the art such as monitoring by hybridization at stringent hybridization conditions or carrying out a conventional PCR reaction under normal conditions without modifications. However, the ability of these tests to detect PCV-2 isolates from different geographic regions is not known. The data described herein show that PCV-2 isolates from different geographic regions vary enough in their genomic sequences to indicate that the known techniques will not be consistent in each case and, therefore, the usefulness of the prior methods is questionable.

Lin et al. employed PCR with restriction fragment length polymorphism (RFLP) for typing avian infectious bronchitis virus (IBV) (Z. Lin et al., “A new typing method for the avian infectious bronchitis virus using polymerase chain reaction and restriction fragment length polymorphism,” Arch. Virol. 116:19-31 (1991)). Jackwood et al. (U.S. Pat. No. 6,214,538) also describe methods of distinguishing between serotypes of avian IBV based on certain restriction fragment length polymorphism (RFLP) patterns after amplifying the S1 glycoprotein gene region of IBV via PCR. However, the methods of Lin et al. and Jackwood et al. would not work to differentiate PCV isolates because the PCR-RFLP tests were preferentially designed and nucleotide sequence-specific for IBV. There have been no reports of the feasibility of applying PCR and RFLP to distinguish PCV infections in pigs.

The value of diagnosing PCV-2 infections and studying the pathogenesis of PCV-2 using PCR and other molecular approaches depends on the knowledge of the extent of genetic variation among PCV-2 isolates from different geographic regions. In addition, the development of an effective vaccine against PMWS also requires a better understanding of the extent of genetic variation among PCV-2 isolates, which has heretofore not been shown. Thus, a universal and more sensitive PCR assay that can detect both PCV-1 and PCV-2 isolates from various geographic regions is needed.

### BRIEF SUMMARY OF THE INVENTION

The present invention concerns a novel method for detecting and differentiating PCV infections in a biological sample taken from a pig which involves amplifying a fragment from an extracted nucleic acid, preferably by PCR; digesting the fragment with a suitable restriction enzyme, preferably with a unique NcoI restriction enzyme; forming a restriction fragment length polymorphism pattern; and then detecting the presence or absence of a PCV isolate. The invention further concerns new oligonucleotide primers for differentiating the PCV infections comprising a nucleotide sequence selected from the group consisting of MCV1 having a nucleotide sequence set forth in SEQ ID NO: 1 and MCV2 having a nucleotide sequence set forth in SEQ ID NO: 2. Moreover, this invention provides a novel kit for detecting and distinguishing PCV infections that includes the new oligonucleotide primers and a suitable restriction enzyme.

### BRIEF DESCRIPTION OF THE DRAWINGS

The background of the invention and its departure from the art will be further described hereinbelow with reference to the accompanying drawings, wherein:

FIG. 1 shows the genome organization of PCV-2. The origin of replication (o), the putative capsid gene (ORF2), the PCR-RFLP fragment, and the two overlapping PCR fragments used to determine the complete genome of PCV-2 are indicated in the circular map. The relative positions of the oligonucleotide primers used herein are indicated by arrows with respective numbers: 1, CV1; 2, CV2; 3, CV3; 4, CV4; 5, CV1-1; 6, CV1-2; 7, CV2-1; 8, CV2-2; 9, CV3-1; 10, CV3-2; 11, CV4-1; 12, CV4-2; 13, MCV1; 14, MCV2. The sequences and designations of these primers are listed in Table 2, below.

FIGS. 2A and 2B illustrate the nucleotide sequence alignment of the region amplified in the PCR-RFLP assay. The regions from which the consensus PCR primers, MCV1 (which corresponds to SEQ ID NO: 1) and MCV2 (which corresponds to SEQ ID NO: 2), were chosen are underlined. The unique NcoI restriction enzyme site that is present in all PCV-2 isolates is indicated by asterisks (*). The sequence of the PCV-2 isolate 26606 is shown on top, and only differences from that sequence are indicated for other isolates. The sequences used in the alignment are cited herein.

FIGS. 3A-3C represent the amino acid sequence alignment of the putative capsid protein (ORF2) of PCV-1 and PCV-2 isolates sequenced thus far. Deletions are indicated by hyphens (-). Amino acid sequence differences are indicated with asterisks (*) above the alignment. The sequences used in the alignment are cited herein.

FIG. 4 displays a phylogenetic tree based on the complete genomic nucleotide sequences of all PCV isolates. The tree was constructed with the aid of the PAUP program (commercially available from David L. Swofford, Smithsonian Institute, Washington, D.C., distributed by Sinauer Associates, Inc., Sunderland, Mass.). Branch-and-bound searching and midpoint rooting options were used to produce a consensus tree. A scale bar that represents the numbers of character-state changes is shown. Branch lengths are proportional to the numbers of character state changes. The geographic locations of the isolates are also indicated with the usual state abbreviations and the following country abbreviations: CAN, Canada; TAI, Taiwan; FR, France; GER, Germany; IRE, Ireland. BOV stands for a bovine strain from Canada that was reported by Nayar et al. (G. P. Nayar et al., 1999, supra).

FIGS. 5A and 5B provide the detection and differentiation of PCV infections by a PCR-RFLP assay. The top panel (FIG. 5A) shows the results of PCR amplification of a 243 bp fragment from tissue samples containing PCV isolates (both PCV-1 and PCV-2) but not from a negative control liver tissue sample (lane 1). The bottom panel (FIG. 5B) shows the results of RFLP analysis of the PCR products in the following lanes: L, 50 bp DNA ladder; lane 1, a sample of liver tissue from a control specific pathogen-free (SPF) pig; lanes 2 to 11, tissue samples from 10 pigs with PMWS; lane 12, PK-15 cells containing PCV-1 (ATCC accession number CCL-33); lane 13, a sample containing both PCV-1 and PCV-2. The expected PCR fragment (FIG. 5A) and three RFLP fragments of 243 bp, 168 bp and 75 bp, respectively (FIG. 5B), are indicated with arrows.

### DETAILED DESCRIPTION OF THE INVENTION

In accordance with the present invention, a new differential polymerase chain reaction-restriction fragment length polymorphism (PCR-RFLP) assay for detecting and distinguishing between infections in pigs caused by the nonpathogenic type-1 porcine circovirus (PCV-1) and the pathogenic PMWS-associated type-2 porcine circovirus (PCV-2) is described. Generally, the method comprises the steps of extracting nucleic acid, preferably DNA, from a biological sample taken from a pig; amplifying a fragment from the extracted nucleic acid, preferably by PCR; digesting the fragment with a suitable restriction enzyme, preferably with a unique NcoI restriction enzyme; forming an RFLP pattern; and then detecting the presence or absence of a PCV isolate by observing the presence or absence of an oligonucleotide fragment selected from the group consisting of approximately 243 base pairs, approximately 168 base pairs, approximately 75 base pairs and a combination thereof. The invention further provides new oligonucleotide primers for differentiating the PCV infections comprising a nucleotide sequence selected from the group consisting of MCV1 having a nucleotide sequence set forth in SEQ ID NO: 1 and MCV2 having a nucleotide sequence set forth in SEQ ID NO: 2.

Based upon the genetic characterization of field isolates of PCV-2 and sequence alignment of all PCV-1 and PCV-2 isolates available, a set of consensus PCR primers is selected from two conserved regions of the PCV genome to amplify a fragment of approximately 243 bp for both PCV-1 and PCV-2 isolates. To test the feasibility of these primers in amplifying both PCV-1 and PCV-2 isolates from clinical samples, DNA is extracted from tissue samples of the six PMWS cases in which the PCV-2 genomic sequences have been determined. DNA is also extracted from tissue samples of four additional cases of PMWS from Iowa in which the PCV-2 sequences had not yet been determined. DNA extracted from the PK-15 cell line (ATCC accession number CCL-33) is used as the source of PCV-1. DNA extracted from a sample of liver tissue collected from a specific pathogen-free pig is used as a negative control. A fragment of the PCV genome is amplified from the tissue samples of all ten cases of PMWS as well as from the PCV-1 contaminated PK-15 cells.

By utilizing a unique NcoI restriction enzyme site that is present only in the sequences of the PCV-2 isolates, the PCR-RFLP assay of the present invention differentiates between infections caused by PCV-1 and PCV-2. After digestion of the PCR products with NcoI, the resulting RFLP patterns revealed that all products amplified from PCV-2 isolates produced two fragments of approximately 168 bp and approximately 75 bp each, whereas the PCR product amplified from PCV-1 produced only the undigested fragment of approximately 243 bp. DNA extracted from a sample mixed with both PCV-2 (from diseased pig assigned serial identification number 40860) and PCV-1 (PK-15 cells) is also subjected to PCR amplification. After digestion with the NcoI restriction enzyme, the PCR product amplified from the mixed sample produced three fragments of approximately 243 bp, approximately 168 bp and approximately 75 bp, respectively. Thus, the PCR-RFLP assay of the present invention is able to detect clinical samples having potential dual infection caused by PCV-1 and PCV-2. The uniqueness of this diagnostic test is that it will not only detect PCV infection but it will also differentiate between infections caused by nonpathogenic PCV-1 and pathogenic PMWS-associated PCV-2 from different geographic regions in the world.

Preferably, the differential diagnostic test for PCV according to the present invention is employed as a qualitative PCR-RFLP assay. However, with slight modification known to those of ordinary skill in the art, one can utilize the PCR-RFLP assay to quantitate the porcine circovirus as well. As a quantitative PCR assessment, the quantitation may be expressed as genome equivalent (GE) of PCV DNA per milliliter of sample.

Suitable sources of the nucleic acid that can be used in the assay include, but are not limited to, single or double stranded DNA and the like. The diagnostic test can readily be performed using any typical clinical or biological sample, such as, for example, liver, spleen, tonsil, lymph nodes, bile, feces, serum, plasma, etc. The nucleic acid may be extracted from the clinical or biological sample by well-known techniques (see, for example, Maniatis et al., “Molecular Cloning: A Laboratory Manual,” pp. 280-281, Cold Spring Harbor Laboratory, Cold Spring Harbor, Mass. (1982)).

The techniques of PCR and RFLP as applied to the present invention are also well-known to those of ordinary skill in the art (PCR2: A Practical Approach, Ed. M. J. McPherson et al., IRL Press, Oxford, N.Y., 1995). The preferred method of amplifying the targeted sequences for detection of the PCV isolate is PCR, which has been described in detail in U.S. Pat. Nos. 4,965,188; 4,800,159; 4,683,195; 4,683,202, among numerous others. While either high or low stringency conditions may be used, the PCR of this invention is preferably performed under relatively high stringency conditions. Low stringency conditions have a possible risk of yielding non-specific amplified products (the so-called “high background”). Instead of PCR, other well-known amplification methods such as the ligase chain reaction (LCR), the nucleic acid sequence-based amplification (NASBA), the strand displacement amplification (SDA), etc. can be utilized in the practice of this invention although less conveniently.

Generally speaking, RFLP is often used in the art to identify a certain pattern in the lengths of restriction fragments after digestion of a nucleic acid molecule with a specific restriction endonuclease and is well-known for identifying sources of genetic material through variations in gene sequences. RFLP has also been described in U.S. Pat. Nos. 4,965,188; 4,800,159 and several other patents.

Six type-2 porcine circovirus isolates (PCV-2) are genetically characterized from cases of confirmed PMWS in different geographic regions of North America. The extent of genetic variation among these six PCV-2 isolates and all other known PCV isolates (both PCV-1 and PCV-2) is analyzed. Based on the data generated, the universal PCR-RFLP assay of the present invention is developed and able to detect and differentiate between infections with PCV-1 and PCV-2 in pigs from different geographic regions.

To determine the extent of genetic heterogeneity of PCV-2 isolates, the complete genome of six PCV-2 isolates from different regions of North America are amplified by PCR and sequenced. Sequence and phylogenetic analyses confirmed that two distinct genotypes of PCV exist: the nonpathogenic PCV-1 and PMWS-associated PCV-2. However, within the major genotype of PCV-2, several minor branches that have been identified appear to be associated with geographic origins. Two French PCV-2 isolates diverge the most in their genomic sequences from other PCV-2 isolates and form a distinct branch. Other minor but distinguishable branches have also been identified for a Taiwan PCV-2 isolate, two of the Canadian PCV-2 isolates. All the U.S. PCV-2 isolates are closely related but the Canadian isolates vary, to some extent, in their genomic sequences. The data described herein indicate that, although the genome of PCV-2 is generally stable among different isolates, PCV-2 isolates from different geographic regions vary in their genomic sequences. This variation may have important implications for PCV-2 diagnosis and research. Based on the genetic analyses of available PCV strains, the universal PCR-RFLP assay is developed to discern between infections with nonpathogenic PCV-1 and pathogenic PCV-2.

PCV-2 is readily detectable from pigs with PMWS in different regions of the North America, but porcine reproductive and respiratory syndrome virus (PRRSV) antigen is also detected in most of the PMWS cases (Table 1, below). The extent of genetic variation of PCV-2 isolated from different geographic regions of the U.S. is not known since only a few PCV-2 isolates from the U.S. have been genetically characterized (B. M. Meehan et al., 1998, supra; I. Morozov et al., 1998, supra). Thus, the genetic characterization involved six North American isolates of PCV-2 (one Canadian and five U.S. isolates) from pigs with PMWS in different geographic regions. Sequence analysis of the complete genome and of the putative capsid gene ORF2 indicated that these six North American isolates of PCV-2 are closely related to other known PCV-2 isolates worldwide. The putative capsid gene (ORF2) of PCV is highly variable and displays as low as a 90% sequence identity among certain PCV-2 isolates. Despite the overall heterogeneic nature of ORF2, the N-terminal region of ORF2 among all PCVs is highly conserved and possesses a high percentage of basic amino acids, suggesting that the amino terminal region of the putative capsid protein may have DNA binding activity and may be in contact with the PCV DNA in the native virion (F. D. Niagro et al., “Beak and feather disease virus and porcine circovirus genomes: intermediates between the geminiviruses and plant circoviruses,” Arch. Virol. 143:1723-1744 (1998)). Phylogenetic analysis revealed that all PCV-2 isolates sequenced thus far form a major genotype, whereas all PCV-1 isolates are closely related and form another genotype. Based on the phylogenetic analysis, it is evident that both PCV-1 and PCV-2 evolved from the same ancestor, but they may have undergone divergent evolution. Gibbs et al. analyzed the genomes of circoviruses and plant nanoviruses and showed that circoviruses most likely evolved from a plant nanovirus (M. J. Gibbs et al., “Evidence that a plant virus switched hosts to infect a vertebrate and then recombined with a vertebrate-infecting virus,” Proc. Natl. Acad. Sci. USA 96:8022-8027 (1999)). It is believed that the plant nanovirus switched hosts to infect a vertebrate and then recombined with a vertebrate-infecting virus (id.). Within the major genotype of PCV-2, several minor branches are also identified. The two French PCV-2 isolates (AF055393 and AF055394) diverge the most from all other PCV-2 isolates. The clinical significance of this divergence is not known. LeCann et al. reported that a PCV1-like virus was isolated from pigs with wasting disease in France, but they failed to experimentally reproduce the disease with this isolate (P. LeCann et al., “Piglet wasting disease,” Vet. Rec. 141:660 (1997)). Phylogenetically, all the U.S. PCV-2 isolates sequenced thus far are closely related. However, genetic variation is observed among the Canadian PCV-2 isolates. Two of the Canadian isolates, AF109398 and AF117753, form a minor branch separating from other Canadian or U.S. isolates. Two other Canadian isolates, 34464 sequenced herein and AF109399, also differ phylogenetically from the U.S. and other Canadian PCV-2 isolates. A PCV-2 isolate from Taiwan (AF116528) also forms a distinguishable minor branch. The origin of the bovine circovirus isolate is not known, but its close genetic relatedness with PCV-2 suggested that the bovine circovirus may be of swine origin, and that cross-species infection of PCV between bovine and swine is possible. These data suggest that, although the genome of PCV-2 is relatively stable in general, minor genetic differences do exist among PCV-2 isolates from different geographic regions.

PMWS is an emerging and economically important swine disease. The PMWS-associated PCV-2 is genetically related to the nonpathogenic PCV-1, which is ubiquitous in the pig population. The differential diagnoses of PCV-1 and PCV-2 infections as provided herein are very important. The assay of this invention uniquely differentiates between infections in pigs caused by nonpathogenic PCV-1 and pathogenic PMWS-associated PCV-2. By doing so, the assay is better than standard procedures by being useful in xenotransplantation in which there is zero tolerance for circovirus infection. Therefore, the availability of the differential diagnostic test of the present invention will tremendously benefit the swine industry and medical treatments dependent upon healthy pig tissues.

Advantageously, the differential PCR-RFLP diagnostic test of this invention is rapid, sensitive, easy-to-perform and specific for the porcine circoviruses and its associated disease. As such, this diagnostic test is reliable in screening for both PCV-1 and PCV-2 infection in pigs and, particularly, in xenograft donor pigs. Because the data described herein show that PCV-2 isolates from different geographic regions vary to some extent in their genomic sequences, the ability of the typical tests known in the art, such as PCR, IHC and in situ hybridization, to detect PCV-2 isolates from different geographic regions would not be reliable. In sharp contrast, the universal PCR-RFLP assay of this invention provides an improved method that can detect and discern all PCV isolates. The term “universal” in the context of the present invention means that the assay can potentially detect all PCV isolates since the primers are uniquely based upon all known isolates. The universal assay may detect more genetically-divergent isolates than the conventional tests. Moreover, because the novel PCR-RFLP assay is based upon the nucleotide sequences of both PCV-1 and PCV-2, it not only detects but differentiates PCV-1 and PCV-2, as well. Because the sample PCV isolates can come from various geographic regions, the assay of the present invention has broader applicability. The assay is beneficial in that it is more sensitive and reliable than the earlier attempts to detect PCV-2.

A further embodiment of the present invention provides a standardized PCR-RFLP assay kit for detecting and distinguishing PCV infections. It is contemplated that the assay kit can be made in large scale commercial units and marketed to Veterinary Diagnostic Laboratories to screen for infections caused by PCV-1 and PCV-2 in pigs, porcine tissues, DNA extracts from pigs and the like. The kit, for example, may contain the new MCV1 and MCV2 primers, appropriate reactants including a suitable restriction enzyme such as the unique NcoI restriction enzyme and a sample showing the RFLP pattern of the restriction fragments of PCV-1 and PCV-2 for comparison purposes. The primers may be the natural oligonucleotides derived directly from the isolated or extracted DNA, cloned cDNA by methods known in the art or synthetic products.

Based on the genetic analyses of all PCV isolates, the universal PCR-RFLP assay of the present invention is able to diagnose PCV-2 infection and to differentiate between infections with PCV-1 and PCV-2 in pigs. This assay utilizes a pair of PCR primers selected from two conserved regions of PCV-1 and PCV-2 genome and a unique NcoI restriction enzyme site, which is now found to exist only in pathogenic PCV-2 isolates and to be absent in nonpathogenic PCV-1. The universal PCR-RFLP assay of this invention is the first reported instance to notice and make use of the unique NcoI site and the NcoI restriction enzyme according to the method of the invention described herein. It is appreciated, however, that other restriction endonucleases may substitute for NcoI and yield marginal to satisfactory results at a different cleavage site of the DNA sequence.

The feasibility of this PCR-RFLP to detect PCV-2 and to differentiate between PCV-2 and PCV-1 is validated by using clinical samples of confirmed PMWS cases collected in different geographic regions, and a sample intentionally mixed with both PCV-1 and PCV-2. The results indicate that this PCR-RFLP is accurate and fast in diagnosing PCV-2 infection from PMWS cases of different geographic regions, in differentiating infections between PCV-1 and PCV-2 and in detecting dual infection with PCV-1 and PCV-2. This universal PCR-RFLP assay will help clinicians diagnose PMWS cases associated with PCV-2 infection in different geographic regions of the world, and will also be useful for screening xenograft donor pigs and confirming that the pigs are free of circovirus infection. In sum, this PCR-RFLP assay is useful for studying the pathogenesis of PCV-2, detecting PCV-2 infection in pigs from different geographic regions and screening donor pigs for use in xenotransplantation.

The complete genomic (nucleotide) sequences of the six PCV-2 isolates sequenced for the method of the present invention have been deposited with the Genbank database and have been made publicly available since Jul. 23, 2000 under accession numbers AF264038, AF264039, AF264040, AF264041, AF264042 and AF264043.

The following examples demonstrate certain aspects of the present invention. However, it is to be understood that these examples are for illustration only and do not purport to be wholly definitive as to conditions and scope of this invention. It should be appreciated that when typical reaction conditions (e.g., temperature, reaction times, etc.) have been given, the conditions both above and below the specified ranges can also be used, though generally less conveniently. The examples are conducted at room temperature (about 23° C. to about 28° C.) and at atmospheric pressure. All parts and percents referred to herein are on a weight basis and all temperatures are expressed in degrees centigrade unless otherwise specified.

A further understanding of the invention may be obtained from the non-limiting examples that follow below.

### EXAMPLE 1

### Isolation of DNA from Tissue Samples

Various tissue samples (liver, spleen, tonsil, lymph nodes, etc.) were collected from pigs with PMWS as confirmed by immunohistochemistry (IHC). The tissues were stored until use at −80° C. The complete PCV-2 genome was amplified, sequenced and characterized from tissue samples of six selected PMWS cases originated from different geographic regions of North America: two cases from Utah, one from Missouri, one from Iowa, one from Illinois, and one from Canada (Table 1, below). These six PMWS cases, along with four more field cases of PMWS (Table 1, below) from Iowa, were also characterized by the PCR-RFLP analyses.

DNA was extracted from the various tissue samples with a QIAamp DNA Mini kit (Qiagen, Inc., Valencia, Calif.) according to the protocol supplied by the manufacturer. For each DNA extraction, 25 mg of tissue samples were used. The resulting DNA was eluted in DNase, RNase and proteinase-free water (Eppendorf 5 Primer, Inc., Boulder, Colo.).

TABLE 1
Porcine circovirus isolates used in the present invention and previously reported

| | | Geographic | IHC^{a} | IHC | ISH^{b} | Clinical | Histopathological | |
|---|---|---|---|---|---|---|---|---|
| Type | ID | location | PRRSV | PCV | PCV | signs^{c} | lesions^{d} | References |
| PCV-2 | 26606^{e} | UT | + | ND | + | Resp | Pneumonia, | this study |
| | | | | | | | lymphoid depletion, | |
| | | | | | | | enteritis | |
| | 26607^{e} | UT | + | ND | + | Resp, | Pneumonia, | this study |
| | | | | | | diarrhea | enteritis, hepatitis, | |
| | | | | | | | nephritis | |
| | 40856^{e} | MO | + | + | ND | Resp, | Pneumonia, | this study |
| | | | | | | wasting | lymphoid depletion, | |
| | | | | | | | hepatitis, nephritis | |
| | 40895^{e} | IA | − | + | ND | Resp, | Pneumonia, | this study |
| | | | | | | wasting | lymphoid depletion | |
| | 34464^{e} | Canada | + | + | ND | Resp | Pneumonia | this study |
| | 10489^{e} | IL | − | + | ND | Resp, | Pneumonia, | this study |
| | | | | | | wasting | lymphoid depletion | |
| | 38835 | IA | + | + | ND | Resp | Pneumonia, | this study |
| | | | | | | | lymphoid depletion | |
| | 36688 | IA | + | + | ND | Resp, | Pneumonia, | this study |
| | | | | | | wasting, | lymphoid depletion, | |
| | | | | | | dermatitis | nephritis, dermatitis | |
| | 40860 | IA | + | + | ND | Resp | Pneumonia | this study |
| | 40887 | IA | + | + | ND | Resp | Pneumonia | this study |
| | AF055391 | CA | | | | | | A |
| | AF027217 | CA | | | | | | B |
| | AF109397 | France^{f} | | | | | | C, GenBank |
| | AJ223185 | IA | | | | | | D |
| | AF055394 | France | | | | | | A |
| | AF085695 | Canada | | | | | | GenBank |
| | AF086836 | Canada | | | | | | GenBank |
| | AF086835 | Canada | | | | | | GenBank |
| | AF086834 | Canada | | | | | | GenBank |
| | AF112862 | Canada | | | | | | GenBank |
| | AF166528 | Taiwan | | | | | | GenBank |
| | AF109399 | Canada | | | | | | GenBank |
| | AF109398 | Canada | | | | | | GenBank |
| | AF117753 | Canada | | | | | | GenBank |
| | AF055393 | France | | | | | | A |
| | AF055392 | Canada | | | | | | A |
| PCV-1 | AF071879 | PK15 cell | | | | | | E |
| | Y09921 | Germany | | | | | | F |
| | U49186 | Ireland | | | | | | G |
| | AF012107 | France | | | | | | F |

^{a}IHC: immuohistochemistry;
^{b}ISH: in situ hybridization
^{c}Clinical signs: Resp = respiratory disease, wasting = anorexia and weight loss
^{d}Histopathological lesions: Pneumonia = interstitial pneumonia
^{e}PCV-2 isolates sequenced in connection with the present invention;
^{f}Bovine isolate
References: A = B. M. Meehan et al., 1998, supra; B = A. L. Hamel et al., 2000, supra; C = P. LeCann et al., 1997, supra; D = I. Morozov et al., 1998, supra; E = F. D. Niagro et al., 1998, supra; F = A. Mankertz et al., 1997, supra; G = B. M. Meehan et al., 1997, supra

### EXAMPLE 2

### PCR Amplification of the Complete Genome of PCV-2

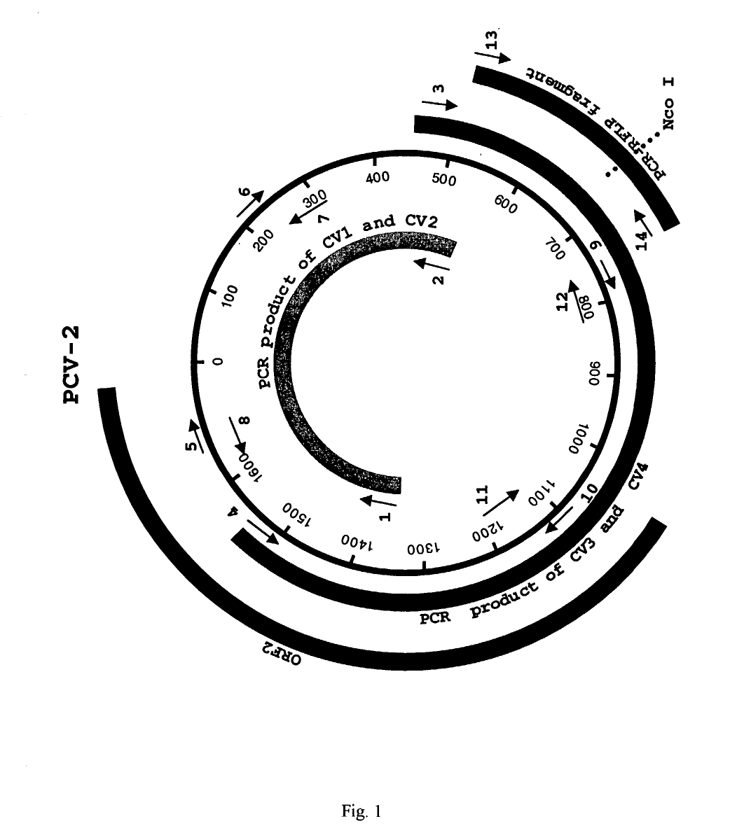

Two sets of PCR primers were designed on the basis of the published PCV-2 sequence. These primers amplify two overlapping fragments that represent the entire genome of PCV-2 (FIG. 1). The first set of primers, CV1 and CV2 (Table 2, below), amplifies a 989 bp fragment, and the second set of primers, CV3 and CV4 (Table 2, below), amplifies a 1092 bp fragment. The extracted DNA was amplified by PCR using AmpliTaq Gold polymerase (Perkin Elmer, Norwalk, Conn.). The PCR reaction consisted of 35 cycles of denaturation at 94° C. for 1 min, annealing at 55° C for 1 min, and extension at 72° C. for 3 min, followed by a terminal extension at 72° C. for 7 min.

### EXAMPLE 3

### Nucleotide Sequencing Sequence and Phylogenetic Analyses

The PCR products of expected sizes were purified by electrophoresis on a 1% agarose gel followed by extraction with a Geneclean Kit (Bio101, La Jolla, Calif.). Both strands were sequenced with a variety of sequencing primers (Table 2, below) with an ABI automated DNA Sequencer at Virginia Tech's DNA Sequencing Facility. The sequences of the primers used to sequence the complete genome of PCV-2 are listed in Table 2, below and their relative positions in the circular genome are indicated (FIG. 1). The sequences were compiled and analyzed by the MacVector program (commercially available from Oxford Molecular Ltd., Beaverton, Oreg.). The percentages of sequence identity among different PCV isolates were determined with the Clustal alignment program in the MacVector package. Sequence alignments were performed with the ALIGN program in the MacVector package. Phylogenetic analyses were conducted with the aid of the PAUP program (commercially available from David L. Swofford, Smithsonian Institute, Washington, D.C., distributed by Sinauer Associates, Inc., Sunderland, Mass.). Branch-and-bound searching and midpoint rooting options were used to produce a consensus tree.

TABLE 2
Oligonucleotide primers used in
the present invention

| | | Appli- | Posi- |
|---|---|---|---|
| ID | Primer Sequence | cation | tion^{b} |
| CV 1 | 5′-AGGGCTGTGGCCTTTGTTAC-3′ | PCR, | 1336- |
| | (corresponds to SEQ ID NO:3) | Seq^{a} | 1356 |
| CV 2 | 5′-TCTTCCAATCACGCTTCTGC-3′ | PCR, | 536- |
| | (corresponds to SEQ ID NO:4) | Seq | 556 |
| CV 3 | 5′-TGGTGACCGTTGCAGAGCAG-3′ | PCR, | 453- |
| | (corresponds to SEQ ID NO:5) | Seq | 475 |
| CV 4 | 5′-TGGGCGGTGGACATGATGAG-3′ | PCR, | 1523- |
| | (corresponds to SEQ ID NO:6) | Seq | 1544 |
| CV 1-1 | 5′-GAGGATCTGGCCAAGATGGCTG-3′ | Seq | 1674- |
| | (corresponds to SEQ ID NO:7) | | 1695 |
| CV 1-2 | 5′-AGGACGAACACCTCACCTCCAG-3′ | Seq | 213- |
| | (corresponds to SEQ ID NO:8) | | 234 |
| CV 2-1 | 5′-GCAGCGGGCACCCAAATACCAC-3′ | Seq | 279- |
| | (corresponds to SEQ ID NO:9) | | 300 |
| CV 2-2 | 5′-ACGTATCCAAGGAGGCGTTACC-3′ | Seq | 1718- |
| | (corresponds to SEQ ID NO:10) | | 1739 |
| CV 3-1 | 5′-AGACTAAAGGTGGAACTGTACC-3′ | Seq | 770- |
| | (corresponds to SEQ ID NO:11) | | 791 |
| CV 3-2 | 5′-TTGTACATACATGGTTACACGG-3′ | Seq | 1083- |
| | (corresponds to SEQ ID NO:12) | | 1104 |
| CV 4-1 | 5′-TGTGGACCACGTAGGCCTCGGC-3′ | Seq | 1146- |
| | (corresponds to SEQ ID NO:13) | | 1167 |
| CV 4-2 | 5′-TGGTAATCAGAATACTGCGGGC-3′ | Seq | 799- |
| | (corresponds to SEQ ID NO:14) | | 820 |
| MCV 1 | 5′-GCTGAACTTTTGAAAGTGAGCGGG-3′ | PCR- | 508- |
| | (corresponds to SEQ ID NO:1) | RFLP | 517 |
| MCV 2 | 5′-TCACACAGTCTCAGTAGATCATCCCA- | PCR- | 725- |

3′

| | | |
|---|---|---|
| (corresponds to SEQ ID NO:2) | RFLP | 750 |

^{a}Seq: primers used for DNA sequencing to determine the complete genome of PCV-2
^{b}The relative positions of these oligonucleotide primers are indicated in FIG. 1.

### EXAMPLE 4

### Development of a PCR-RFLP Assay

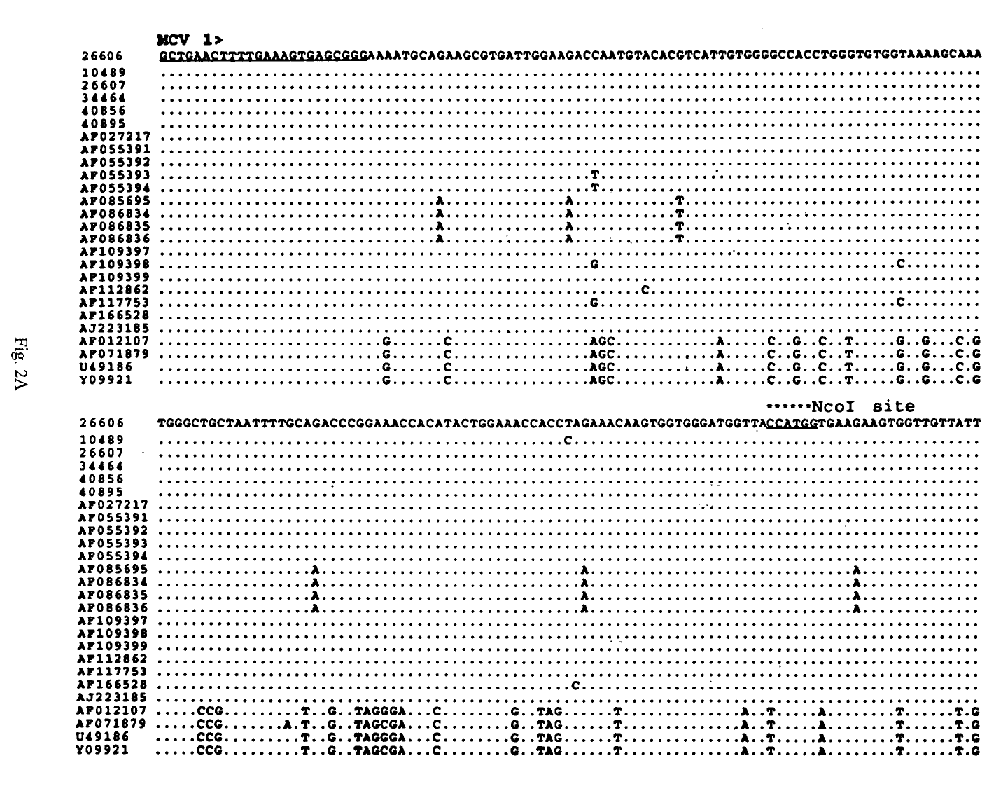

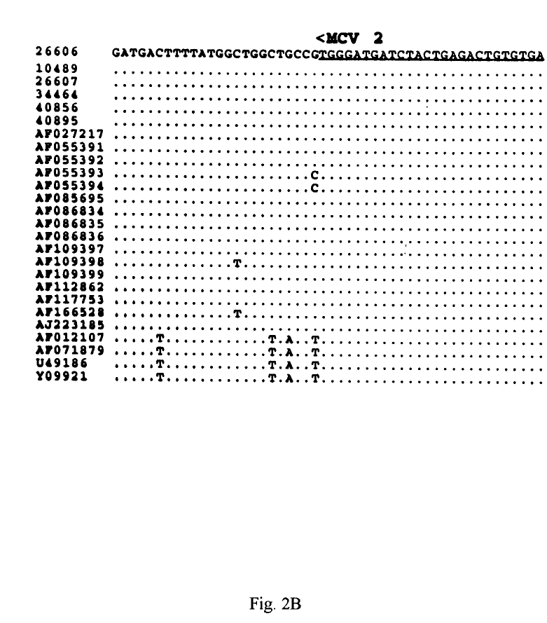

A PCR-RFLP assay was developed to differentiate between strains of PCV-1 and PCV-2 infecting pigs. Briefly, the complete sequences of the six PCV-2 isolates determined herein and the complete sequences of all other PCV sequences available in the GenBank (both PCV-1 and PCV-2) were aligned with the Clustal program. Based on this alignment, a set of conserved PCR primers (MCV 1 and MCV 2, Table 2, above) was designed to amplify a fragment of 243 bp from samples containing either PCV-1 or PCV-2 or both. The sequences of the two chosen PCR primers are identical among all of the known PCV-1 and PCV-2 isolates including the six PCV-2 isolates sequenced herein (FIGS. 2A-2B). The PCR reaction consisted of 37 cycles of denaturation at 94° C. for 1 min., annealing at 56° C. for 1 min. and extension at 72° C. for 1.5 min. The amplified PCR products were subsequently digested with a unique restriction enzyme, NcoI, which is present in all PCV-2 isolates but not in PCV-1 isolates (FIGS. 2A-2B). The digested PCR products are separated on a 2% agarose gel for RFLP analysis.

### EXAMPLE 5

### Genetic Characterization of PCV-2 Isolates from Pigs with PMWS in Different Geographic Regions

To determine the extent of genetic heterogeneity among PCV-2 isolates, the complete genome of PCV-2 was amplified and sequenced from one case of PMWS in Canada (serial identification number 34464) and five cases of PMWS in the U.S.: two cases from Utah (serial identification numbers 26606 and 26607), one from Missouri (serial identification number 40856), one from Iowa (serial identification number 40895), and one from Illinois (serial identification number 10489). The PMWS cases used in this invention possessed clinical signs consistent with PMWS (Table 1, above) and were confirmed to be positive for PCV-2 antigen by IHC. All six of these PMWS cases are negative for swine influenza virus, but four of the six cases are found positive for porcine reproductive and respiratory syndrome virus (PRRSV) antigen (Table 1, above).

The genomic DNA of PCV-1 isolates ranges from 1758 to 1760 bp in length. Sequence analyses of the complete genome of six PCV-2 isolates from this study showed that, like all other PCV-2 isolates, the complete genome of these six PCV-2 isolates is 1768 bp in length. All the PCV-2 isolates sequenced are closely related to each other, displaying 95 to 99% nucleotide sequence identity (Table 3, below). Two French PCV-2 isolates, AF055393 and AF055394, displayed the most sequence divergence from other PCV-2 isolates, ranging from 95 to 96% identity. Similarly, the four PCV-1 isolates sequenced thus far (AF071879, Y09921, U49186, AF012107) are closely related to each other and share 98 to 99% nucleotide sequence identity in the entire genome (Table 3, below). Moreover, the nucleotide sequence identity between PCV-1 and PCV-2 is only about 75 to 77% for the entire genome.

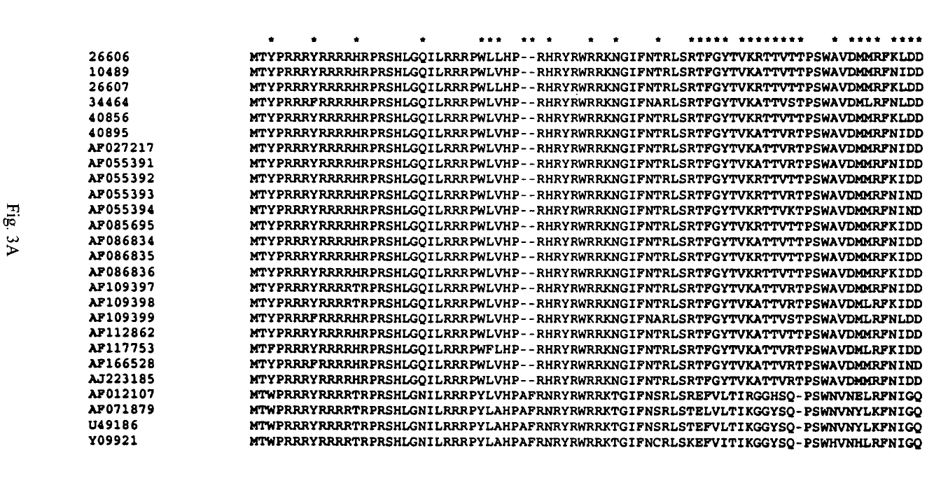

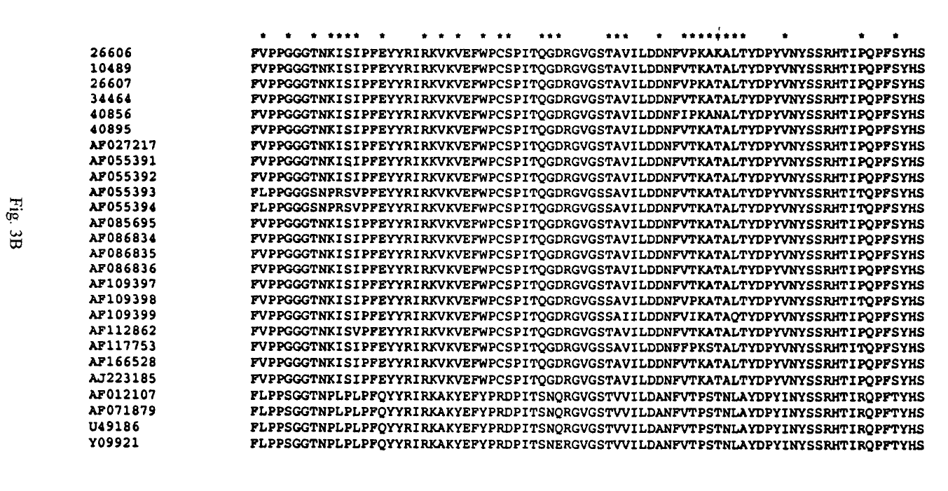

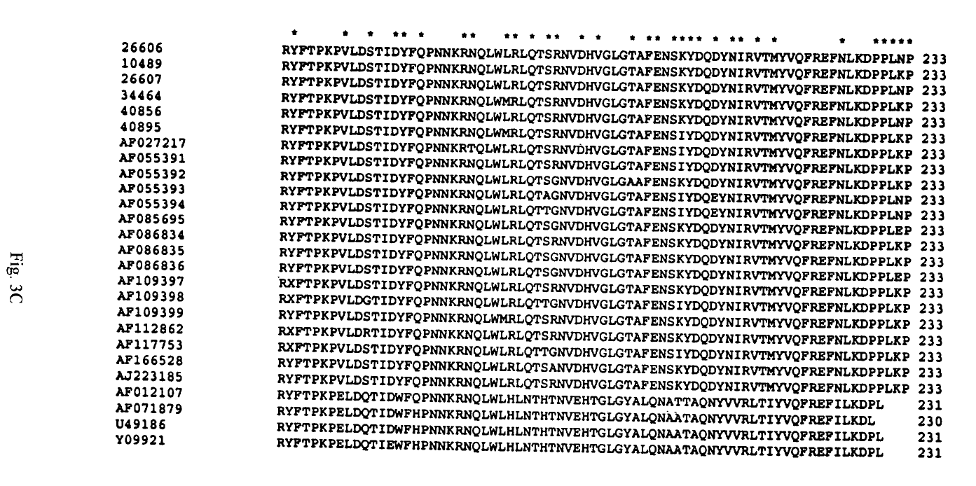

The open reading frame 2 (ORF2) of PCV is believed to code for the putative capsid protein (A. Mankertz et al., 1997, supra; J. Mankertz et al., 1998, supra; F. D. Niagro et al., 1998, supra). Sequence analysis indicated that the ORF2 of PCV-1 isolates encodes for a protein of 230 to 231 amino acid residues, whereas the ORF2 of PCV-2 isolates encodes for a protein of 233 amino acid residues (FIGS. 3A-3C). Pairwise sequence comparisons revealed that the ORF2 of all PCV-2 isolates shared 91 to 100% nucleotide sequence and 90 to 100% amino acid sequence identity (Table 3, below). The two French isolates, AF055393 and AF055394, have only about 90 to 93% nucleotide sequence identity with other PCV-2 isolates (Table 3, below). The four PCV-1 isolates share 97 to 99% nucleotide sequence and 94 to 98% amino acid sequence identity in the ORF2. Between PCV-1 and PCV-2 isolates, there exists only 65 to 67% nucleotide sequence and 63 to 68% amino acid sequence identity in the ORF2 (Table 3, below). However, sequence analysis revealed that the N-terminal region of the ORF2 is very rich in basic amino acid residuals (arginine and lysine) and is highly conserved among PCV, both PCV-1 and PCV-2 isolates (FIGS. 3A-3C).

TABLE 3
Pairwise comparison of the complete genomic and putative capsid gene (ORF2) sequences
of porcine circovirus type-1 and type-2

| | 26606 | 10489 | 26607 | 40895 | 34464 | 40856 | AF085695 | AF086834 | AF086835 |
|---|---|---|---|---|---|---|---|---|---|
| 26606 | | 98/96 | 99/99 | 98/95 | 96/95 | 99/98 | 98/97 | 98/96 | 98/97 |
| 10489 | 99^{a} | | 98/97⁷ | 99/98 | 97/97 | 98/96 | 98/98 | 99/100 | 99/98 |
| 26607 | 99 | 99 | | 98/96 | 96/95 | 99/98 | 98/97 | 98/97 | 99/97 |
| 40895 | 99 | 99 | 99 | | 97/97 | 98/95 | 98/96 | 99/98 | 98/97 |
| 34464 | 98 | 98 | 98 | 98 | | 96/95 | 96/95 | 97/97 | 97/96 |
| 40856 | 99 | 99 | 99 | 98 | 98 | | 98/97 | 98/96 | 98/97 |
| AF085695 | 98 | 98 | 98 | 98 | 97 | 98 | | 99/98 | 99/99 |
| AF086834 | 98 | 98 | 98 | 98 | 97 | 98 | 99 | | 99/98 |
| AF086835 | 98 | 98 | 98 | 98 | 97 | 98 | 99 | 99 | |
| AF086836 | 98 | 98 | 98 | 98 | 97 | 98 | 99 | 98 | 99 |
| AF109398 | 96 | 96 | 96 | 96 | 96 | 96 | 95 | 95 | 95 |
| AF109399 | 97 | 97 | 97 | 97 | 98 | 97 | 96 | 96 | 96 |
| AF112862 | 98 | 99 | 98 | 99 | 98 | 98 | 98 | 98 | 98 |
| AF117753 | 96 | 96 | 96 | 96 | 96 | 95 | 95 | 95 | 95 |
| AF027217 | 99 | 99 | 99 | 99 | 98 | 99 | 98 | 98 | 98 |
| AF055391 | 99 | 99 | 99 | 99 | 98 | 99 | 98 | 98 | 98 |
| AF055392 | 99 | 99 | 99 | 99 | 98 | 99 | 99 | 98 | 98 |
| AF055393 | 95 | 95 | 95 | 95 | 95 | 95 | 95 | 95 | 95 |
| AF055394 | 95 | 96 | 95 | 96 | 95 | 95 | 95 | 95 | 95 |
| AF109397 | 99 | 99 | 99 | 99 | 98 | 99 | 98 | 98 | 98 |
| AF166528 | 97 | 97 | 97 | 97 | 97 | 97 | 96 | 97 | 96 |
| AJ223185 | 99 | 99 | 99 | 99 | 98 | 98 | 98 | 98 | 98 |
| AF012107 | 76 | 77 | 76 | 76 | 76 | 76 | 76 | 76 | 76 |
| AF071879 | 76 | 76 | 76 | 76 | 76 | 76 | 76 | 75 | 76 |
| U49186 | 76 | 76 | 76 | 76 | 76 | 76 | 76 | 76 | 76 |
| Y09921 | 76 | 76 | 76 | 76 | 76 | 76 | 76 | 76 | 76 |
| | AF086836 | AF109398 | AF109399 | AF112862 | AF117753 | AF027217 | AF055391 | AF055392 | AF055393 |
| 26606 | 98/97 | 93/93 | 94/93 | 97/95 | 93/93 | 98/95 | 98/95 | 98/96 | 92/92 |
| 10489 | 98/98 | 94/94 | 95/95 | 98/98 | 93/93 | 99/98 | 99/98 | 98/98 | 92/93 |
| 26607 | 98/97 | 93/93 | 94/93 | 97/95 | 93/93 | 98/96 | 98/96 | 98/97 | 92/92 |
| 40895 | 98/96 | 94/95 | 95/95 | 98/96 | 93/93 | 99/99 | 99/99 | 98/96 | 92/93 |
| 34464 | 96/95 | 94/93 | 97/97 | 96/95 | 93/92 | 97/96 | 97/96 | 96/95 | 92/91 |
| 40856 | 98/97 | 93/93 | 94/93 | 97/95 | 92/92 | 98/95 | 98/95 | 98/96 | 92/92 |
| AF085695 | 100/100 | 94/94 | 94/93 | 97/96 | 93/93 | 98/96 | 98/96 | 99/99 | 92/93 |
| AF086834 | 99/98 | 94/94 | 95/95 | 98/98 | 93/93 | 99/98 | 99/98 | 99/98 | 92/93 |
| AF086835 | 99/99 | 94/95 | 94/93 | 98/96 | 93/93 | 98/97 | 98/97 | 99/99 | 93/93 |
| AF086836 | | 94/94 | 94/93 | 97/96 | 93/93 | 98/96 | 98/96 | 99/99 | 92/93 |
| AF109398 | 95 | | 93/92 | 94/94 | 97/96 | 94/95 | 94/95 | 94/94 | 93/93 |
| AF109399 | 96 | 96 | | 94/93 | 93/91 | 95/94 | 95/94 | 94/93 | 91/90 |
| AF112862 | 97 | 96 | 97 | | 93/92 | 98/96 | 98/96 | 98/96 | 92/92 |
| AF117753 | 95 | 97 | 96 | 96 | | 93/93 | 93/93 | 93/93 | 91/91 |
| AF027217 | 98 | 96 | 97 | 99 | 96 | | 99/99 | 98/96 | 93/93 |
| AF055391 | 98 | 96 | 97 | 99 | 96 | 99 | | 98/96 | 93/93 |
| AF055392 | 98 | 96 | 97 | 99 | 96 | 99 | 99 | | 93/93 |
| AF055393 | 95 | 95 | 95 | 95 | 95 | 96 | 96 | 96 | |
| AF055394 | 95 | 95 | 95 | 95 | 95 | 96 | 96 | 96 | 99 |
| AF109397 | 98 | 97 | 97 | 99 | 96 | 99 | 99 | 99 | 95 |
| AF166528 | 97 | 96 | 96 | 97 | 95 | 97 | 97 | 97 | 96 |
| AJ223185 | 98 | 96 | 97 | 99 | 96 | 99 | 99 | 99 | 95 |
| AF012107 | 76 | 76 | 76 | 76 | 76 | 76 | 76 | 76 | 77 |
| AF071879 | 76 | 76 | 76 | 76 | 75 | 76 | 76 | 76 | 76 |
| U49186 | 76 | 76 | 76 | 76 | 75 | 76 | 76 | 76 | 76 |
| Y09921 | 76 | 76 | 76 | 76 | 75 | 76 | 76 | 76 | 76 |

| | AF055394 | AF109397 | AF166528 | AJ223185 | AF012107 | AF071879 | U49186 | YO9921 |
|---|---|---|---|---|---|---|---|---|
| 26606 | 92/92 | 97/95 | 95/95 | 98/96 | 66/66 | 65/64 | 65/65 | 65/65 |
| 10489 | 92/93 | 99/98 | 96/98 | 99/99 | 67/68 | 66/66 | 66/66 | 66/66 |
| 26607 | 92/92 | 98/96 | 95/95 | 98/96 | 66/66 | 65/64 | 66/65 | 66/65 |
| 40895 | 92/93 | 99/98 | 96/97 | 99/99 | 66/67 | 66/65 | 66/66 | 66/66 |
| 34464 | 92/91 | 97/96 | 96/96 | 97/97 | 66/67 | 65/65 | 65/66 | 65/66 |
| 40856 | 92/92 | 97/95 | 95/95 | 98/96 | 66/66 | 65/63 | 65/64 | 65/64 |
| AF085695 | 92/93 | 98/96 | 96/96 | 98/97 | 66/67 | 66/65 | 66/66 | 66/66 |
| AF086834 | 93/93 | 99/98 | 96/98 | 99/99 | 67/68 | 66/66 | 66/66 | 66/66 |
| AF086835 | 93/93 | 98/97 | 96/97 | 98/98 | 67/67 | 66/65 | 66/66 | 66/66 |
| AF086836 | 92/93 | 98/96 | 96/96 | 98/97 | 66/67 | 66/65 | 66/66 | 66/66 |
| AF109398 | 93/93 | 95/96 | 93/94 | 94/95 | 67/67 | 66/65 | 66/66 | 66/66 |
| AF109399 | 91/90 | 95/94 | 94/94 | 95/95 | 66/65 | 65/63 | 65/63 | 65/63 |
| AF112862 | 92/92 | 98/97 | 95/96 | 98/97 | 67/67 | 66/65 | 66/66 | 66/66 |
| AF117753 | 92/91 | 93/93 | 93/93 | 93/93 | 66/66 | 65/64 | 65/65 | 65/65 |
| AF027217 | 93/93 | 99/98 | 96/97 | 99/99 | 66/67 | 66/65 | 66/66 | 66/66 |
| AF055391 | 93/93 | 99/98 | 96/97 | 99/99 | 66/67 | 66/65 | 66/66 | 66/66 |
| AF055392 | 93/93 | 98/96 | 96/96 | 98/97 | 66/67 | 66/65 | 66/66 | 66/66 |
| AF055393 | 99/99 | 92/93 | 93/93 | 92/93 | 67/68 | 66/66 | 66/66 | 66/66 |
| AF055394 | | 92/92 | 93/93 | 92/93 | 67/68 | 66/66 | 66/66 | 66/66 |
| AF109397 | 95 | | 96/97 | 99/99 | 67/68 | 66/66 | 66/66 | 66/66 |
| AF166528 | 96 | 97 | | 96/98 | 66/67 | 65/65 | 65/66 | 65/66 |
| AJ223185 | 95 | 99 | | 97 | 66/68 | 66/66 | 66/66 | 66/66 |
| AF012107 | 77 | 77 | 76 | 76 | | 97/95 | 97/96 | 97/94 |
| AF071879 | 76 | 76 | 76 | 76 | 98 | | 99/98 | 98/95 |
| U49186 | 77 | 77 | 76 | 76 | 98 | 99 | | 98/96 |
| Y09921 | 76 | 76 | 76 | 76 | 98 | 99 | 99 | |

^{a}The values in the table are percentage identity of amino acid or nucleotide sequences. The nucleotide sequence comparisons of the complete genomes are presented in the lower left half. The four PCV-1 isolates (AF012107, AF071879, U49186, Y09921) are highlighted with bold face.
^{b}The putative capsid (ORF2) gene is shown at the upper right: nucleotide/amino acid.

### EXAMPLE 6

### Phylogenetic Analysis of PCV-1 and PCV-2 Isolates from Different Geographic Regions Worldwide

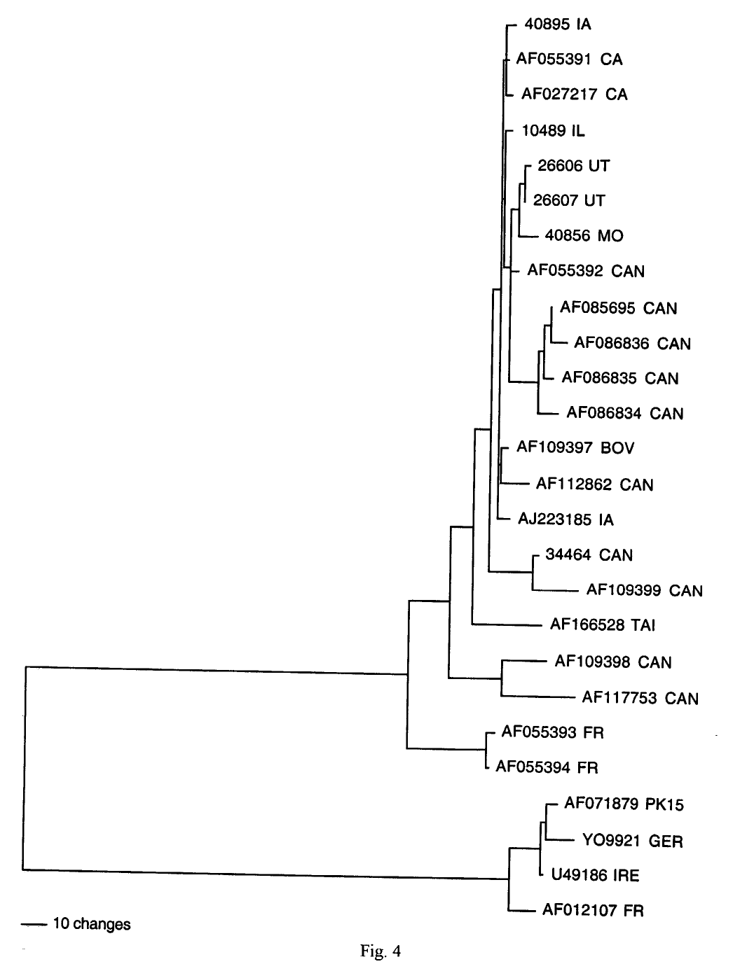

To gain a better understanding of the genetic relationship and evolution of PCV, phylogenetic analyses were performed based on the complete genomic sequences of 26 PCV isolates (both PCV-1 and PCV-2) worldwide, including the six North American PCV-2 isolates sequenced in the present illustration of the invention (FIG. 4). These sequences were either published (A. L. Hamel et al., 1998, supra; A. L. Hamel et al., 2000, supra; A. Mankertz et al., 1997, supra; J. Mankertz et al., 1998, supra; A. Mankertz et al., 2000, supra; B. M. Meehan et al., 1997, supra; B. M. Meehan et al., 1998, supra; I. Morozov et al., 1998, supra; G. P. Nayar et al., 1999, supra; F. D. Niagro et al., 1998, supra; D. Todd et al., 1991, supra) or are available in GenBank (Table 1, above). Phylogenetic analysis confirmed that two distinct genotypes of PCV exist: PCV-1 and PCV-2 (FIG. 4). All 22 PCV-2 isolates are clustered together and form one distinct branch. Similarly, all the four PCV-1 isolates are closely related and form another branch. Within the major genotype of PCV-2, a few minor branches were identified and some of these minor branches appear to be associated with geographic origins of the isolates. All the PCV-2 isolates from different geographic regions of the U.S., which are presently sequenced herein, are grouped closely with other U.S. and most of the Canadian PCV-2 isolates (FIG. 4). The Canadian isolate 34464 sequenced herein is closely related to another Canadian isolate, 109399, but is less related to the U.S. and other Canadian isolates. Two other Canadian isolates, AF109398 and AF117753, form a distinguishable branch and are distantly related to other Canadian and U.S. isolates. An isolate of PCV-2 from Taiwan, AF166526, is clustered within the North American PCV-2 isolates but forms a single minor branch. The two French isolates of PCV-2, AF055393 and AF055394, are closely related to each other but diverge the most from North American PCV-2 isolates. Interestingly, a bovine isolate of circovirus is most closely related to the U.S. isolates of PCV-2.

### EXAMPLE 7

### Development of a PCR-RFLP Assay To Diagnose PCV-2 Infection and to Differentiate Infections Between PCV-1 and PCV-2

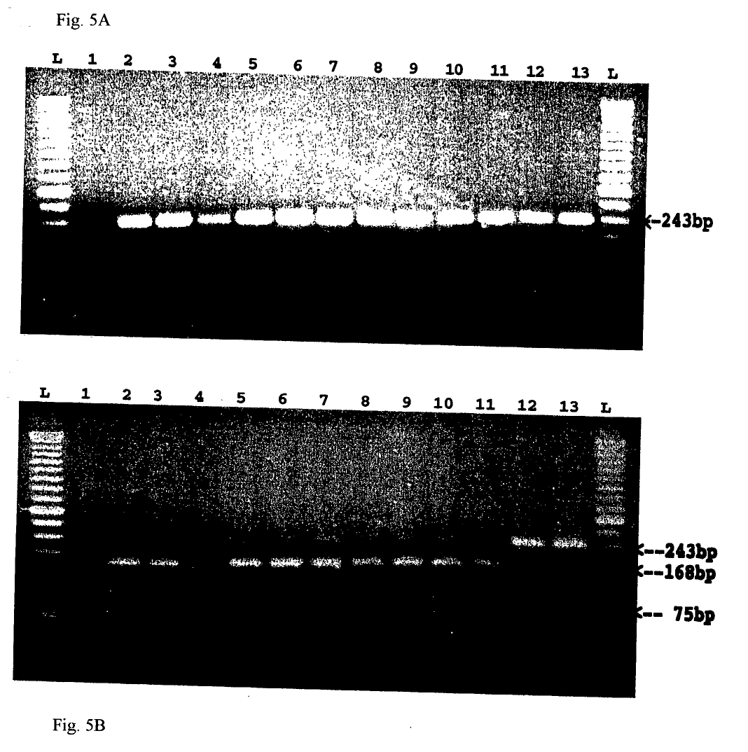

Based on the sequence alignment of all PCV-1 and PCV-2 isolates sequenced thus far, a set of consensus PCR primers was selected from two conserved regions of PCV genome to amplify a fragment of 243 bp for both PCV-1 and PCV-2 isolates (FIG. 5A). To test the feasibility of these primers in amplifying both PCV-1 and PCV-2 isolates from clinical samples, DNA was extracted from tissue samples of the six PMWS cases in which the PCV-2 genomic sequences have been determined. DNA was also extracted from tissue samples of four additional cases of PMWS from Iowa (Table 1, above), and the PCV-2 sequence from these four cases of PMWS has not been determined. DNA extracted from the PK-15 cell line (ATCC accession number CCL-33) was used as the source for PCV-1. DNA extracted from a sample of liver tissue collected from a specific pathogen-free pig was used as a negative control. An expected fragment of PCV genome was amplified from tissue samples of all 10 cases of PMWS as well as from the PCV-1 contaminated PK-15 cells. By utilizing a unique restriction enzyme site (NcoI) that is present only in the sequences of PCV-2 isolates (FIGS. 2A-2B), a PCR-RFLP assay was utilized to differentiate between infections with PCV-1 and PCV-2. After digestion of the PCR products with NcoI, the resulting RFLP patterns revealed that all products amplified from PCV-2 isolates produced two fragments of 168 bp and 75 bp each, whereas the PCR product amplified from PCV-1 produced only the undigested fragment of 243 bp (FIGS. 5A and 5B). DNA extracted from a sample mixed with both PCV-2 (from diseased pig assigned serial identification number 40860) and PCV-1 (PK-15 cells) was also subjected to PCR amplification. After digestion with NcoI restriction enzyme, the PCR product amplified from the mixed sample produced 3 fragments of 243 bp, 168 bp and 75 bp, respectively (FIG. 5B). Thus, this PCR-RFLP assay is able to detect clinical samples with potential dual infection with PCV-1 and PCV-2.

In the foregoing, there has been provided a detailed description of particular embodiments of the present invention for the purpose of illustration and not limitation. It is to be understood that all other modifications, ramifications and equivalents obvious to those having skill in the art based on this disclosure are intended to be included within the scope of the invention as claimed.

What is claimed is:

1. A method for detecting and differentiating porcine circovirus (PCV) infections, which comprises the steps of:
a. extracting nucleic acid from a biological sample taken from a pig;
b. amplifying a fragment from the extracted nucleic acid;
c. digesting the amplified fragment with a restriction enzyme;
d. forming a restriction fragment length polymorphism (RFLP) pattern from an undigested or digested fragment; and
e. detecting the presence or absence of a PCV isolate.

2. The method according to claim 1, wherein the biological sample is liver, spleen, tonsil, lymph node, bile, feces, serum or plasma.

3. The method according to claim 2, wherein the nucleic acid is DNA.

4. The method according to claim 3, wherein the step of amplifying the fragment from the extracted DNA is performed by a polymerase chain reaction (PCR).

5. The method according to claim 4, wherein the PCR employs an oligonucleotide primer selected from the group consisting of MCV1 having a nucleotide sequence set forth in SEQ ID NO: 1 and MCV2 having a nucleotide sequence set forth in SEQ ID NO: 2.

6. The method according to claim 5, wherein the restriction enzyme is NcoI.

7. The method according to claim 6, wherein the step of detecting the presence or absence of the PCV isolate comprises observing the presence or absence of an oligonucleotide fragment selected from the group consisting of approximately 243 base pairs, approximately 168 base pairs, approximately 75 base pairs and a combination thereof.

8. The method according to claim 7, wherein the step comprises observing the presence of an undigested oligonucleotide fragment of approximately 243 base pairs to confirm a nonpathogenic PCV-1 infection.

9. The method according to claim 7, wherein the step comprises observing the presence of two oligonucleotide fragments of approximately 168 base pairs and approximately 75 base pairs to confirm a pathogenic PCV-2 infection.

10. The method according to claim 7, wherein the step comprises observing the presence of an undigested oligonucleotide fragment of approximately 243 base pairs and two oligonucleotide fragments of approximately 168 base pairs and approximately 75 base pairs to confirm the presence of PCV-1 and PCV-2 infections.

11. The method according to claim 7, wherein the step comprises observing the absence of the oligonucleotide fragment to confirm the absence of a PCV infection in the pig.

12. An oligonucleotide primer for differentiating PCV infections, which comprises a nucleotide sequence selected from the group consisting of MCV1 having a nucleotide sequence set forth in SEQ ID NO: 1 and MCV2 having a nucleotide sequence set forth in SEQ ID NO: 2.

13. An assay kit for detecting and differentiating PCV infections, which comprises:
a. an oligonucleotide primer selected from the group consisting of MCV1 having a nucleotide sequence set forth in SEQ ID NO: 1 and MCV2 having a nucleotide sequence set forth in SEQ ID NO: 2; and
b. a restriction enzyme.

14. The assay kit according to claim 13, wherein the restriction enzyme is NcoI.

15. The assay kit according to claim 14, which further comprises a sample RFLP pattern of the restriction fragments of PCV-1 and PCV-2 for comparison.
